(19) United States
(12) Patent Application Publication
(10) Pub. No.: US 2008/0161820 A1
(43) Pub. Date: Jul. 3, 2008

(54) METHOD AND APPARATUS FOR LOCATING A STARTING POINT FOR A SURGICAL PROCEDURE

(75) Inventors: Michael A. Wack, Warsaw, IN (US); Rebecca L. Parrott, Winona Lake, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER TECHNOLOGY, INC., Warsaw, IN (US)

(21) Appl. No.: 11/619,076

(22) Filed: Jan. 2, 2007

(51) Int. Cl. A61B 17/90

(52) U.S. Cl. 606/96

(57) ABSTRACT

A device for locating a site in anatomy for a surgical procedure.

### BACKGROUND AND SUMMARY

The present invention relates to systems for fixing bone, and, more particularly, to an instrument for identifying the location on the tissue for placement of bone fasteners.

According to one embodiment, the present disclosure provides a guide assembly comprising a radiolucent body having a distal end, a proximal end, a longitudinal axis, and a plurality of bores extending from the proximal end to the distal end parallel to the longitudinal axis; and at least one pin sized to be received in one of the plurality of bores in the radiolucent body. The at least one pin is further sized to extend out of the one of the plurality of bores and into the anatomy of a patient.

Another embodiment of the present disclosure provides a guide comprising: a body having a distal end, a proximal end, a longitudinal axis, and a plurality of bores extending from the proximal end to the distal end parallel to the longitudinal axis, the distal end conforming to anatomy of a patient; and a handle coupled the body.

Yet another embodiment of the present disclosure provides a guide including: a handle; and means for positioning a second pin in a femur relative to a first pin in the femur.

Another embodiment of the present disclosure provides a method of locating a desired point in anatomy including the steps of: placing a radiolucent locator in contact with the anatomy; coupling a first reference member to the radiolucent locator and the anatomy; determining a position of the desired point relative to the first reference member; and coupling a second reference member to the radiolucent locator and the anatomy.

### BRIEF DESCRIPTION OF THE DRAWINGS

The above-mentioned and other features of the disclosure, and the manner of attaining them, will become more apparent and better understood by reference to the following description of an embodiment of the disclosure taken in conjunction with the accompanying drawings, wherein:

FIG. 1 is a perspective view of a locator apparatus according to the present disclosure;

FIG. 2 is an end view of the locator apparatus of FIG. 1 positioned on anatomy;

FIG. 3 is a side view of the locator apparatus of FIG. 1 positioned on anatomy and a pin extending through the locator and into the anatomy;

FIG. 4 is a bottom view of the locator apparatus of FIG. 1 and a pair of pins extending through the locator;

FIG. 5 is a perspective view of an alternative second embodiment head for the locator apparatus;

FIG. 6 is another perspective view of the second embodiment head;

FIG. 7 is a perspective view of an alternative third embodiment head for the locator apparatus; and

FIG. 8 is another perspective view of the third embodiment head.

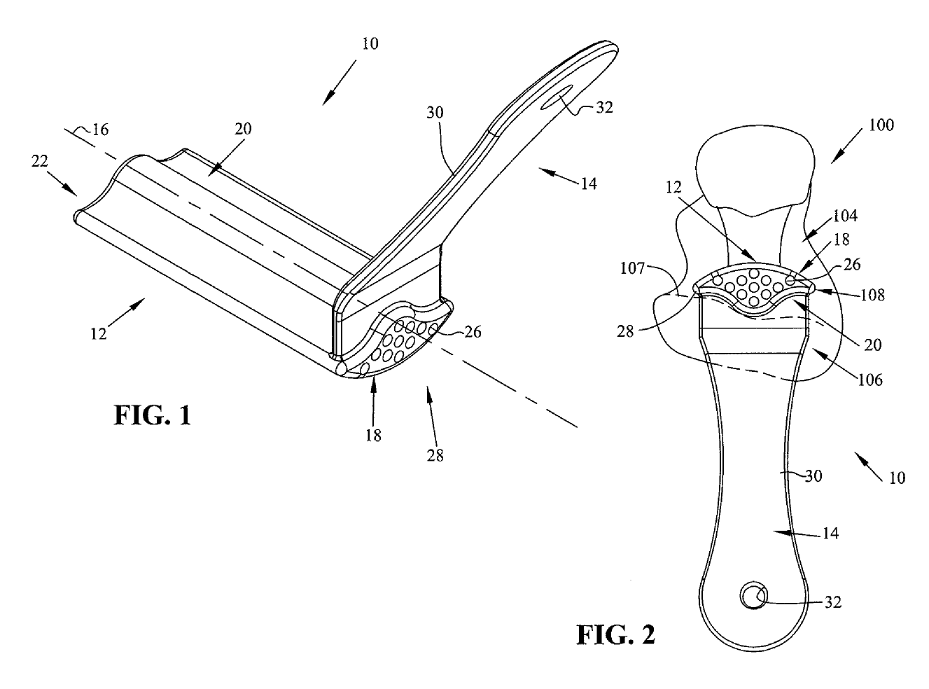

Corresponding reference characters indicate corresponding parts throughout the several views. The exemplification set out herein illustrates one preferred embodiment of the invention, in one form, and such exemplification is not to be construed as limiting the scope of the invention in any manner.

### DETAILED DESCRIPTION OF THE INVENTION

The description that follows refers to an antegrade femoral nail application. While described with respect to an antegrade femoral nail application, the principles of the present disclosure can be applied to other surgical applications.

Intramedullary nails (not shown) are available in a plurality of diameters such that one may be selected that is appropriate for the anatomy of the intended patient. Placement of the nail in a patient presenting a femoral fracture includes several steps, not all of which will be discussed herein, but are known to those of ordinary skill in the art. Before beginning the surgical procedure, the fracture is reduced. Entry into intramedullary canal 102 of femur 100 from proximal end 104, nearest the hip (not shown), may be achieved through greater trochanter 106 or piriformis fossa 108.

For entry through piriformis fossa 108, an incision is made just posterior to midline of trochanter and just medial to prominence of trochanter in trochanteric fossa. Next, a proper entry portal into intramedullary canal 102 is located using first Steinmann pin 110 and one or more radiological views. To aid in locating the proper placement of the entry portal, locator 10 is provided.

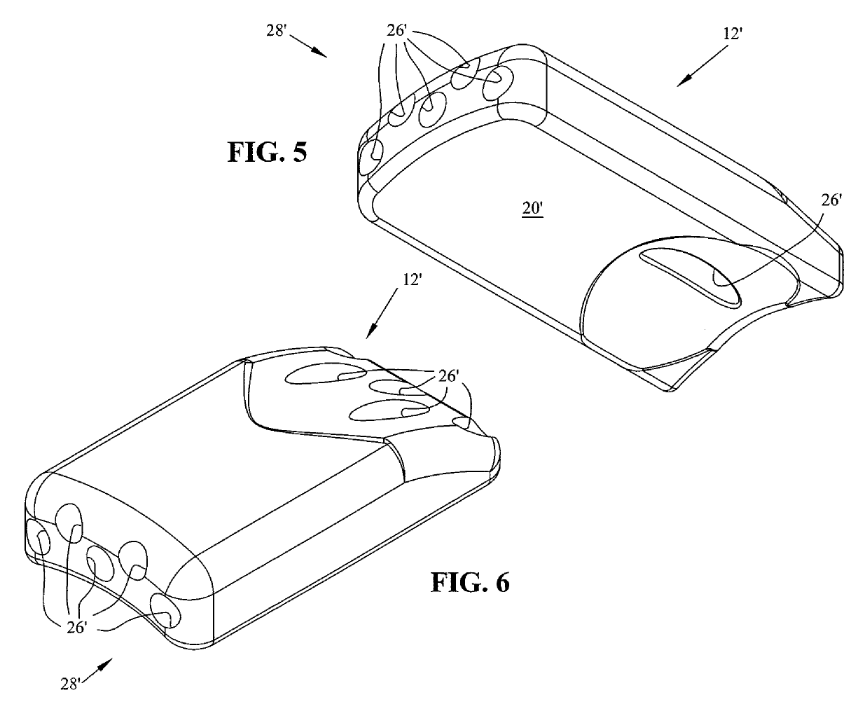

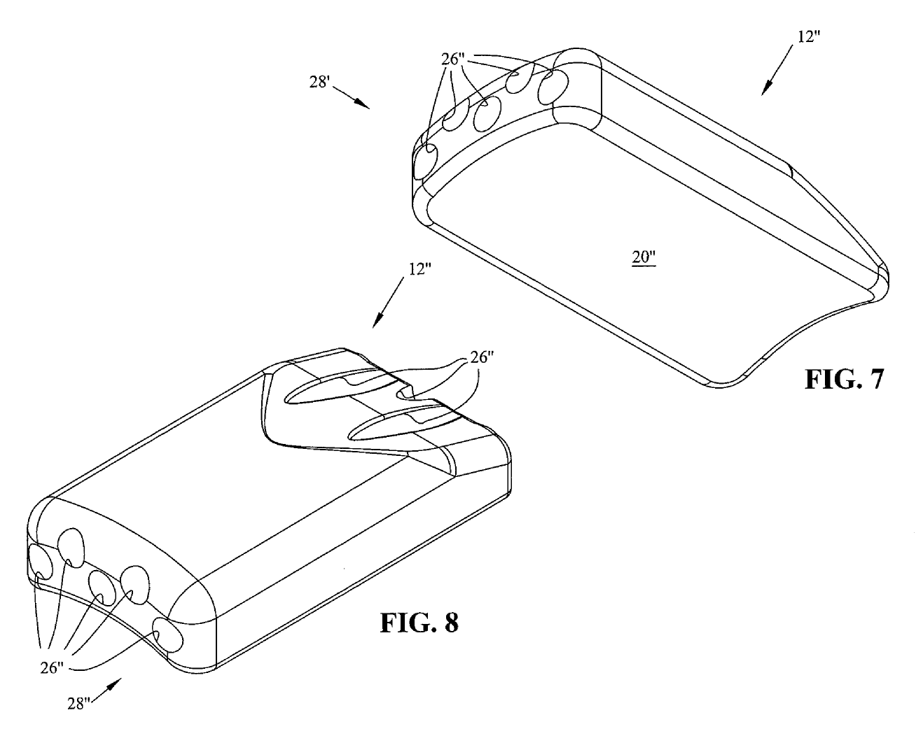

Locator/guide 10 includes body/head 12 and handle 14, both formed from radiolucent material. Head 12 includes arc side 18 and undulating side 20 and defines longitudinal axis 16. Arc side 18 of distal end 22 of head 12 includes cut away portion 24. Arc side 18 and undulating side 20 are shaped to approximate external surfaces of the anatomy surrounding piriformis fossa 108 to allow locator 10 to easily abut piriformis fossa 108 of femur 100. Arc side 18 is shaped to abut inner side 107 of greater trochanter 106. It should be appreciated that head 12 may be alternatively shaped for applications where differently shaped anatomy is expected or desired to be encountered. Such heads 12′, 12″ are shown in FIGS. 5-8 that may better approximate an encountered piriformis fossa and greater trochanter of femur 100.

Head 12 includes a plurality of bores/barrels 26 (26′, 26″ in FIGS. 5-8) therein. Each bore 26 is substantially circular in cross section and has a similar diameter, although other shapes may be used. Bores 26 extend from proximal end 28 of head 12 to distal end 22 of head 12. When discussing parts of locator 10, “proximal” denotes those parts which are nearest a user, such as a surgeon, and “distal” denotes those parts farther from the user. Bores 26 extend parallel to longitudinal axis 16 of head 12. Bores 26 are sized and shaped to receive Steinmann pins 110, 112 therein. Handle 14 couples to undulating side 20 near proximal end 28 of head 12. (Handle 14 attaches to sides 20′, 20″ near proximal ends 28′, 28″ of heads 12′, 12.″) Handle 14 extends away from proximal end 28 of head 12 to provide grip surfaces 30 for the surgeon. Handle 14 includes bore 32 that provides a surface by which to hang locator 10.

In use, the surgeon grips handle 14 and abuts distal end 22 of head 12 to the selected anatomy, such as piriformis fossa 108. First Steinmann pin 110 is placed through a selected bore 26 and advanced into the anatomy of the patient in an attempt to designate a proper entry point to intramedullary canal 102. Head 12 and longitudinally extending bores 26 are of a length to hold first pin 110 in a stable manner.

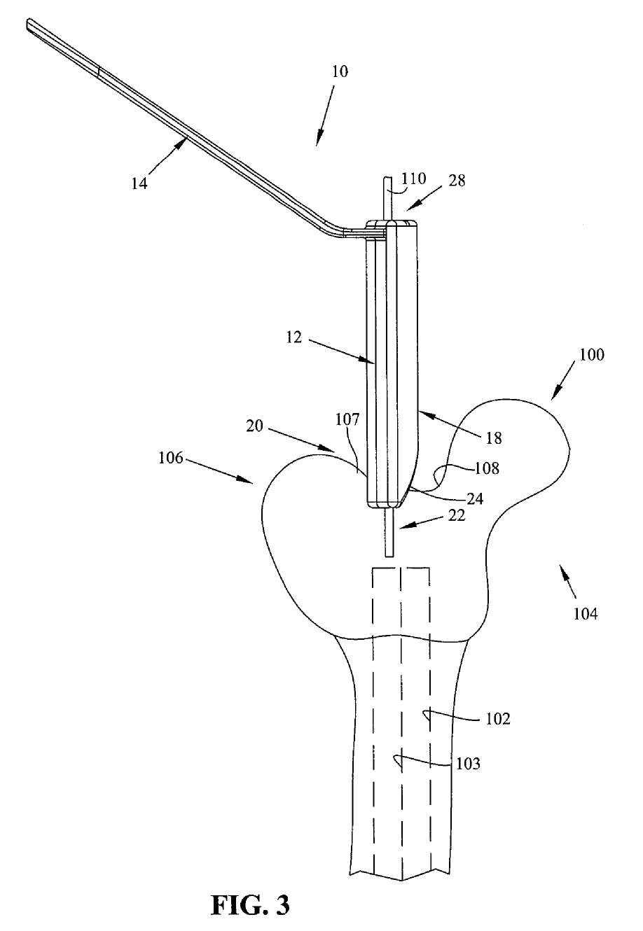

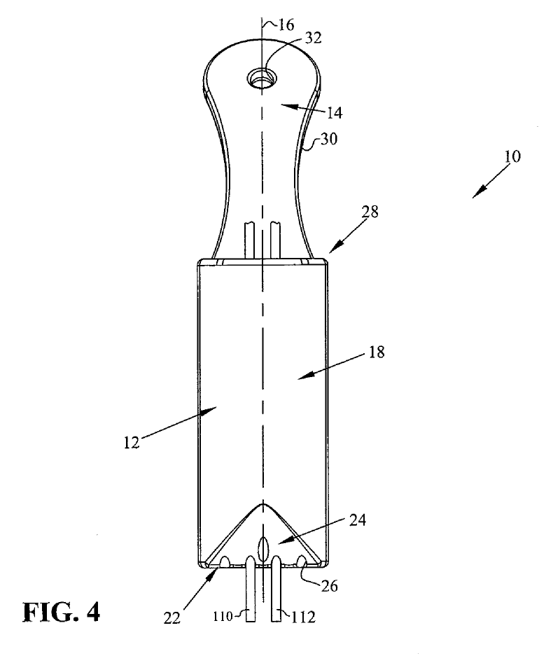

Once placed, as shown in FIG. 3, anterior/posterior and lateral fluoroscopic images are taken of femur 100, locator 10, and first pin 110 in combination. The images show the placement of first pin 110 relative to femur 100 and intramedullary canal 102. Locator 10 does not prevent viewing of first pin 110 and the anatomy on the images due to its radiolucence.

The desired entry portal/starting point should be slightly anterior in piriformis fossa 108. To the extent that first pin 110 is not positioned in the desired starting point, the views are analyzed to determine the offset of the desired location from first pin 110.

The surgeon then determines which bore 26 has an offset from bore 26 engaged by first pin 110 that corresponds to the previously determined offset of the desired location from first pin 110. The surgeon then places second pin 112 into respective bore 26 that is properly offset from the first used bore 26. Anterior/posterior and lateral fluoroscopic images are again taken of femur 100, locator 10, and pins 110, 112 in combination. In this way, pins 110, 112 are utilized as reference members.

The process of taking fluoroscopic views, determining the offset of the desired placement from currently placed pin(s) 110, and placement of subsequent pin 112 is repeated until placement of pin 112 at the desired location is achieved. Once pin 112 is properly located, previously placed pin(s) 110 is removed. If more than two pins 110 are placed during the procedure, certain pins 110 may be removed prior to achieving a properly located pin 112 provided that at least one previously placed pin 110 remains or locator 10 is otherwise fixed relative to the anatomy such that the determined offset can be applied to a fixed reference point.

When the proper position is confirmed, firm pressure is applied on the properly placed pin 112 to further seat pin 112 within the anatomy. Locator 10 is then manipulated to adjust the angle of pin 112 so that it is aligned with intramedullary canal 102. Thus, pin 112 and used bore 26 are substantially co-axial with a longitudinal axis 103 of intramedullary canal 102. The shape of head 12, that conforms to the anatomy, specifically piriformis fossa 108, provides that little or no adjustment of pin angle is likely needed. Further anterior/posterior and lateral views are optionally taken as pin 112 is driven into femur 100. Locator 10 is then removed, leaving pin 112 in place in femur 100.

Then, a reamer (not shown) is placed over pin 112 and utilized to create the entry portal to intramedullary canal 102. Pin 112 and reamer are subsequently removed.

While this invention has been described as having exemplary designs, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A guide assembly comprising:
a radiolucent body having a distal end, a proximal end, a longitudinal axis, and a plurality of bores extending from the proximal end to the distal end parallel to the longitudinal axis; and
at least one pin sized to be received in one of the plurality of bores in the radiolucent body, the at least one pin further sized to extend out of the one of the plurality of bores and into anatomy of a patient.

2. The guide assembly of claim 1, wherein the plurality of bores all have a similar cross-section.

3. The guide assembly of claim 1, further comprising a radiolucent handle coupled to the radiolucent body.

4. The guide assembly of claim 1, wherein the radiolucent body is shaped and sized to conform to anatomy of the patient.

5. The guide assembly of claim 4, wherein the distal end of the radiolucent body conforms to a piriformis fossa.

6. The guide assembly of claim 5, wherein at least one of the plurality of bores centrally aligns with an intramedullary canal when the distal end abuts conforming parts of the piriformis fossa.

7. A guide comprising:
a body having a distal end, a proximal end, a longitudinal axis, and a plurality of bores extending from the proximal end to the distal end parallel to the longitudinal axis, the distal end conforming to anatomy of a patient; and
a handle coupled the body.

8. The guide of claim 7, wherein the body is radiolucent.

9. The guide of claim 7, wherein the distal end of the body conforms to a piriformis fossa.

10. The guide of claim 7, wherein at least one of the plurality of bores is offset from a side of the body by a distance equal to the distance between a longitudinal axis of an intramedullary canal and a greater trochanter of a femur.

11. A guide including:
a handle; and
means for positioning a second pin in a femur relative to a first pin in the femur.

12. The guide of claim 11, wherein the means for positioning is radiolucent.

13. The guide of claim 11, wherein the means for positioning conforms to an external surface of the femur.

14. The guide of claim 11, wherein the means for positioning includes a plurality of bores sized and shaped for receiving the first and second pins.

15. A method of locating a desired point in anatomy including the steps of:
placing a radiolucent locator in contact with the anatomy;
coupling a first reference member to the radiolucent locator and the anatomy;
determining a position of the desired point relative to the first reference member; and
coupling a second reference member to the radiolucent locator and a location of the anatomy substantially the same as the desired point.

16. The method of claim 15, wherein the step of coupling a first reference member to the radiolucent locator includes receiving the first reference member within one of a plurality of bores within the radiolucent locator.

17. The method of claim 15, wherein the step of placing a radiolucent locator includes abutting a portion of the radiolucent locator to a portion of the anatomy to which the radiolucent locator is sized and shaped to conform.

18. The method of claim 15, wherein the step of coupling a first reference member includes placing a first pin through the radiolucent locator and into the anatomy.

19. The method of claim 18, wherein the step of coupling a second reference member includes placing a second pin through the radiolucent locator and into the anatomy.

20. The method of claim 19, wherein the step of coupling a second reference member includes causing the first pin and the second pin to be coupled to the anatomy at the same time.

21. The method of claim 15, wherein the step of determining the position of a desired point relative to the first reference member includes the step of obtaining at least one fluoroscopic image.
